(19) United States
(12) Patent Application Publication
(10) Pub. No.: US 2022/0175225 A1
(43) Pub. Date: Jun. 9, 2022

(54) HYBRID ENDOSCOPE WITH ROTARY DRUM FOR STERILE MEDICAL APPLICATIONS

(71) Applicant: KARL STORZ SE & Co. KG, Tuttlingen (DE)

(72) Inventor: Peter SCHWARZ, Tuttlingen (DE)

(21) Appl. No.: 17/456,713

(22) Filed: Nov. 29, 2021

(30) Foreign Application Priority Data
Dec. 9, 2020 (DE) 10 2020 132 776.5

(51) Int. Cl. A61B 1/00; A61B 1/018; A61B 1/06

(52) U.S. Cl. CPC A61B 1/00105; A61B 1/018; A61B 1/00144; A61B 1/0684; A61B 1/0011

(57) ABSTRACT

Hybrid endoscope, in particular for sterile medical applications, wherein the endoscope comprises a receiving device for an imaging system and a shaft tube, wherein the receiving device is arranged, in a mounted state, on a distal end of the shaft tube. The endoscope is divided into at least two sub-assemblies, wherein a first sub-assembly comprises at least the receiving device, the imaging system and preferably a supply line of the imaging system as re-usable parts, and a second sub-assembly comprises at least the shaft tube as a part for single use, wherein the two sub-assemblies are able to be brought into releasable operative connection by means of an interface.

### TECHNICAL FIELD

The present invention relates to a hybrid endoscope with a number of re-usable parts for sterile medical applications, as per the preamble of claim 1.

### BACKGROUND OF THE INVENTION

Endoscopes, in particular for surgical applications, are cleaned and disinfected for re-use after an operation. The cleaning involves internal line systems, channels and valve connectors and also an imaging system with an associated mechanical adjustment mechanism and with electrical connectors. The disinfection can also entail an autoclaving procedure at high temperatures of over 130° C. and/or the use of chemicals for the disinfection of various infective agents such as bacteria, fungi and viruses. To withstand the adverse conditions during the disinfection, the structural parts of an endoscope are preferably produced from metallic materials which, compared to plastic materials, withstand these conditions for longer and can thus be re-used more often. In particular, the imaging system with electronic connectors is therefore usually inserted into or arranged in a hermetically sealed metal housing.

Following a disinfection process, the cleaned parts are dried and, until subsequent use, are stored in a sterile state and protected from renewed contamination. The storage of re-usable medical endoscopes is regulated under DIN EN 16442, in accordance with which the ambient conditions are to be monitored and the endoscopes are to be stored in special cabinets.

However, despite the time-consuming and laborious disinfection of the endoscope according to regulations, it is not possible to entirely exclude microbial contamination in parts that are particularly difficult to clean, such as the line systems and the long channels of small diameter and the corresponding connectors.

The correct cleaning and the production of hermetically sealed endoscopes are not only associated with higher costs, the function of the endoscope is also greatly limited by the strict design requirements. For example, a field of view of the imaging system is restricted by the arrangement in a rigid housing with glass covers, and a costly curved cover glass is needed. Moreover, on account of stray light on the cover glass, the illumination cannot be pivoted with the imaging system. Furthermore, by being hermetically sealed, the imaging system takes up more room in a shaft tube, such that either an imaging system with low resolution can be used or, alternatively, a small work channel can be used.

As an alternative to disinfectable re-usable endoscopes, it is possible to use single-use endoscopes which are arranged in sterile packaging and can be completely disposed of after use. However, this leads to a large amount of waste material, which needs to be disposed of and has to be correctly treated as special waste.

A shaft or usually a shaft tube of the endoscope can be rigid or flexible or can be flexible in parts. Unless a more detailed description is given, the shaft tube is to be understood below as a rigid and/or flexible shaft tube.

WO 2017/040692 A1 discloses an endoscope with a spherical rotation drum on an elongate rigid shaft tube, especially for use as a disposable instrument, wherein the rotation drum is rotatably mounted in a bearing fork at a distal end of the rigid shaft tube and can be rotated in the interior of the rigid shaft tube by means of a control line. The rotation drum has an imaging system in its interior and is connected to electronic elements in the interior of the rigid shaft tube, wherein a liquid can be guided through the interior of the rigid shaft tube at the same time. In this embodiment of an endoscope with a rotation drum, a majority of the rigid shaft tube is occupied by the electronic elements of the imaging system and of the control line.

Particularly since the electronic elements are routed inside the shaft tube and can there come into contact with liquid from an operation region, aggressive disinfection of these parts is also necessary. Since these parts are not hermetically sealed, for example in a metal housing, the parts cannot be repeatedly cleaned without some risk of damage. Although a disposable instrument can make production and the choice of material easier and can also increase a field of view of the imaging system, high costs can be incurred particularly in replacing the imaging system, the control line and the electronic components.

### SUMMARY OF THE INVENTION

The present invention is therefore based on the object of proposing an endoscope which, while avoiding the problems known from the prior art, permits a large work channel in relation to the internal diameter of the shaft tube, is suitable for sterile medical use, and at the same time permits simple, safe and cost-effective cleaning.

Furthermore, it is an object to specify a method for operating/using the endoscope according to the invention.

This object is achieved by the features of independent claim 1, in respect of the endoscope, and by the features of claim 10, in respect of the method.

Advantageous embodiments are the subject matter of the dependent claims.

According to the invention, a hybrid endoscope, in particular for sterile medical applications, is proposed, wherein the endoscope comprises a receiving device for an imaging system and a shaft tube, wherein the receiving device is arranged, in a mounted state, on a distal end of the shaft tube. The endoscope is here divided into at least two sub-assemblies, wherein a first sub-assembly comprises at least the receiving device, the imaging system and preferably a supply line of the imaging system as re-usable parts, and a second sub-assembly comprises at least the shaft tube as a part for single use, wherein the two sub-assemblies are able to be brought into releasable operative connection by means of an interface.

In this case, the invention has surprisingly recognized that, by using at least two separate sub-assemblies, the parts that are associated with high production costs, in particular the imaging system with high-quality optical and electronic components, can be easily released from a distal end of the shaft tube and can thus be treated in order to be re-used. Low-cost parts, and parts that are simple in terms of their structure but difficult to clean, for example the shaft tube itself, are provided as a further, second sub-assembly for single use. These parts include in particular channels for instrument guidance and a fluid line, and mechanical joints with at least one degree of freedom, which are difficult to hermetically seal or would have to be carefully covered with a metal bellows. In this way, a sterile endoscope can still be used cost-effectively, and even in a way that saves resources, particularly in risky regions where there are multi-resistant microorganisms. Moreover, the separate treatment in particular prevents the formation of pockets of dirt, which could occur during treatment in the assembled state. Furthermore, a high-quality imaging system can be used for precise recording.

In addition to the two sub-assemblies, a division into further structural parts or sub-assemblies would be conceivable, in particular for sections that do not come into direct contact with the operation region and thus undergo less elaborate treatment and in some cases can also be protected against contamination by means of simple and replaceable plastic coverings or have to meet less stringent demands in respect of leaktightness and disinfection.

In a particularly advantageous embodiment of the invention, the supply line of the imaging system runs on an outer face of the shaft tube. In this way, the two sub-assemblies can be particularly easily separated from each other and treated. The possibility of pockets of dirt accumulating in the interior of the shaft tube can be prevented in particular by the supply line being arranged on the outside.

Preferably, the parts of the first sub-assembly are hermetically sealed and therefore autoclavable. In this way, the imaging system can also comprise high-quality components that are able to be re-used and that are not damaged even under aggressive cleaning/disinfection.

According to the teaching of the invention, provision can be made that the receiving device of the first sub-assembly is produced from a metal, and/or the shaft tube of the second sub-assembly is produced from a plastic, and/or the supply line is connected to an autoclavable plug connector. The metallic receiving device of the first sub-assembly can thus withstand the cleaning conditions in an autoclave, in particular with additional chemical cleaning, over a large number of cleaning cycles. Particularly preferably, the shaft tube of the second sub-assembly can be produced cost-effectively from a plastic, for example by injection molding, in large piece numbers. The plug connector is preferably provided as a connector to a central control unit (CCU), which does not come into contact with the operation region or is not contaminated by this.

It is furthermore preferred if the imaging system contains an electronic image recorder and/or an imaging optical unit and/or an illumination device, in particular with LEDs, and/or special assemblies such as laser projectors. In this way, an operation region can be examined and observed. Particularly through the re-usability of the first sub-assembly, high-quality components, in particular for the image recorder, can be installed in the receiving device.

In a preferred embodiment, the receiving device of the first sub-assembly receives the imaging system in a rotation drum, and the second sub-assembly has an interface in a bearing fork in order to bear the rotation drum rotatably.

According to a further preferred embodiment, at least one control line is windable along at least one winding curve preferably on the outer circumference of the rotation drum, in order to pivot the rotation drum.

Further preferably, the supply line of the imaging system is designed as at least one control line of the rotation drum, preferably as a flexible printed circuit board or as a cable, for electronic circuits in the interior of the rotation drum. The supply line can have electronic circuits, in particular of an electronically adjustable imaging optical unit and/or of the electronic image recorder or sensor of the imaging system and/or of the illumination device, preferably with LEDs.

Particularly preferably, the at least one control line is guided in a recess, preferably with a protective collar, in the outer face of the shaft tube, the recess running parallel to the longitudinal axis of the shaft tube. As a result, in the assembled state of the first and second sub-assemblies, the at least one control line is guided in a space-saving and interlocking fashion on the outer face of the shaft tube in order, firstly, to protect the at least one control line from external mechanical influences and in order, secondly, to prevent risk of injury or clamping of tissue during an operation.

Resetting means, preferably a torsion spring at a bearing point of the rotation drum, are arranged on the rotation drum for the purpose of controlling the rotation drum. Here, the torsion spring acts counter to a mechanical moment of the tensioned control line in order to return a pivoted rotation drum to an initial position. Thus, the torsion spring preferably acts counter to an operation of the control line in such a way that a compression force need not be applied to the control line for the purpose of a rotation of the rotation drum, in order to allow the use of a flexible control line.

Alternatively or in addition, the rotation drum can be returnable by a further control line on the opposite side and/or also operable by means of a compression force by way of a dimensionally stable control line, similar to a Bowden cable, or a type of control rod or toothed rack.

Further alternatively, the further control line can preferably be in the form of a wire, wherein a return means, preferably a spring, in particular a helical spring, is preferably fastened in a handle for returning the rotation drum.

Alternatively or in addition, provision can be made that the second sub-assembly has, as interface, a rotatable joint with an adjustment mechanism for fastening the receiving device. Compared to the rotation drum, the receiving device is preferably of a simpler design, preferably not prepared rotatably for a bearing site, and can preferably be connectable at the rear, with a side directed away from the imaging system, to the rotatable joint by means of the interface.

The interface can produce a form-fit and/or force-fit and/or cohesive connection and can preferably be designed as a plug connection or latch connection and/or screw connection.

Provision can preferably be made that the work channel in the shaft tube has at least one fluid line, including preferably an air line and/or a liquid feed line and a liquid extraction line in modular fashion. Especially since the imaging system with the supply line is not arranged within the shaft tube, a plurality of channels, such as the air line together with the liquid feed line and the liquid extraction line, can be provided within the shaft tube in particular. A type of overshaft around the shaft tube for fluid guidance, which increases the external diameter of the shaft tube, is advantageously not required. The functionality of the fluid line can comprise cleaning the optics and/or temperature control and/or cooling of the rotation drum and/or rinsing and insufflating a fluid into an operation region.

Advantageously, the air line is used in a dry operation region in order to dry the rotation drum following a cleaning of the optical unit and/or to cool the rotation drum and the electronic components of the imaging system.

In a dry environment of the endoscope, the work channel preferably has an air line, a liquid feed line and a liquid extraction line as at least one fluid line. The optical unit of the endoscope is cleaned in a dry operation region, preferably in a cleaning position in which the rotation drum is rotated in such a way that the field of view is aligned with the distal end of the shaft tube and the work channel. In this cleaning position, the imaging system can be rinsed with a rinsing liquid from the liquid feed line, wherein the rinsing liquid is removable via the liquid extraction line, and this can minimize rinsing liquid leaking into a dry operation region. The rinsing liquid can preferably be removed from the imaging system using the air line, in order to prevent a disturbance of the field of view of the imaging system by the formation of drops. Further preferably, the liquid feed line, and preferably the air line too, can be embodied as a spray nozzle in order to improve the cleaning effect.

For a watery or wet operation region or environment of the endoscope, the liquid feed line and the liquid extraction line are preferably arranged in the work channel in order to flow around and control the temperature of the rotation drum on the rear side, and in order to rinse the operation region. For rinsing the medical operation region, provision is preferably made for the shaft tube to have holes and/or an opening, preferably a slot-shaped opening, of the work channel on the circumferential side at the distal end, in order to connect the at least one fluid line, preferably the liquid extraction line, to the operation region and in order to remove a rinsing fluid from the operation region. In particular, the shaft tube is open behind the rotation drum in order to ensure a free emergence of the rinsing fluid, in particular of the rinsing water.

Preferably, the work channel of the second sub-assembly is designed to receive at least one instrument and/or contains at least one instrument in order to be able to carry out preferably medical operations in the operation region and, in particular, in the field of view of the imaging system. In this case, the at least one instrument preferably has a controllable tool at the distal end, for example a scissors-type cutting tool or punching tool. The receptacle of the at least one instrument can be provided in addition to or as an alternative to the at least one fluid line. As a result of the external control line and, in particular, the external supply of the imaging system, the relatively large work channel can be utilized for at least one instrument and, at the same time, at least one fluid line.

The at least one instrument can be designed to be likewise separable from the first sub-assembly and, like the first sub-assembly, can be re-used as a further sub-assembly. Alternatively, it is also possible to use disposable instruments, which are not treated or cleaned after use.

In one development, the shaft tube is open at an upper portion, the at least one control line being guided on an opposite lower portion in order preferably to adjust/bend a flexible instrument in a direction perpendicular to the longitudinal axis of the shaft tube and into the open upper portion. The work channel is arranged in the shaft tube and behind the rotation drum in such a way that the flexible instrument slides along the outer circumference of the rotation drum and can be guided through the open upper portion. As a result, the instrument, in particular the functional parts and tools at the distal end of the instrument, can be brought into an operation region, and at the same time the operation region can be tracked by the imaging system within the rotation drum.

Especially for the use of the endoscope with the at least one instrument, provision is preferably made that the rotation drum is fastened to the bearing fork so as to be pivotable about a first rotation axis, and the bearing fork in turn is fastened to the shaft tube in a manner pivotable about a second rotation axis, and the work channel is open along the longitudinal axis in a pivoted state of the bearing fork in order to guide the at least one instrument, in particular, from the work channel into an operation region, the bearing fork preferably being controllable by way of a second control line. The second control line is preferably likewise guided on the outer face of the shaft tube in order not to restrict the work channel in the interior of the shaft tube. Using this embodiment, a rigid instrument in particular is guidable into the operation region in a manner parallel to the longitudinal axis of the shaft tube. Moreover, by rotating the rotation drum about the second rotation axis, a preferably flexible instrument is bendable in a direction perpendicular to the longitudinal axis of the shaft tube.

In addition to the guidance of the at least one instrument, the pivotable bearing fork advantageously facilitates an extended field of view of the imaging system, also for example behind obstacles or corners, or a view along the outer face of the shaft tube. Thus, even the guidance function of the at least one control line along the shaft tube is able to be monitored by means of the imaging system. Furthermore, rinsing or aspiration of fluids from the operation region can be promoted by way of the open work channel.

The pivotable bearing fork can preferably correspond to the already mentioned interface with the rotatable joint with adjustment mechanism, wherein a pivotable receiving device can be fastened to the joint.

Alternatively or in addition, the bearing fork can also be connected to the shaft tube by means of a flexible or elastically deformable element, wherein the flexible element provides a passive restoring force in order to pivot the bearing fork about the second rotation axis and to open the work channel for the instrument.

Preferably, the external diameter of the shaft tube is 3 mm to 6 mm, with the rotation drum not exceeding this external diameter. Such shaft tubes are suitable for a large number of non-invasive medical operations.

In a further preferred embodiment, a plurality of control lines are windable on different winding curves along the circumference of the rotation drum, in particular with different distances from the rotation axis, in order to carry out a different rotational adjustment of the rotation drum in the case of the same adjustment travel of the control line. In particular, the adjustment travel parallel to the longitudinal axis of the shaft tube and the different rotational adjustment can lead to predefined viewing directions of the imaging system or to different rotational speeds, wherein, in the case of the same adjustment travel of the control line, the rotational adjustment reduces with decreasing distance from the first rotation axis of the rotation drum. The tensile force required for the rotational adjustment can also be set by the different distances.

Particularly preferably, the different winding curves are arranged in steps along the first rotation axis on the circumference of the rotation drum, wherein the winding curves are preferably arranged in the direction of the outer sides of the rotation drum with a decreasing distance from the first rotation axis.

The invention also relates to a method for operating and cleaning an above-described endoscope with rotation drum, in particular the field of view of the imaging system, wherein in a first step the rotation drum is rotated from an observation position for an operation region to a cleaning position in which the field of view is aligned with the distal end of the shaft tube and the work channel, and wherein the cleaning method described below is particularly suitable for endoscopes in a dry environment or a dry operation region since leakage of rinsing liquid into a operation region can be minimized. In a second step, the imaging system and/or the imaging optical unit and/or the illumination device is/are rinsed, especially in the field of view, using a preferably aqueous rinsing fluid from at least one fluid line, preferably a liquid feed line with a spray nozzle. Preferably, the aqueous rinsing fluid can be aspirated by means of a further, second fluid line, preferably a liquid extraction line, in order to minimize the leakage of rinsing fluid into an operation region. In the process, the rotation drum can be pivoted in order to clean the entire field of view. In a preferred third step, the preferably aqueous rinsing fluid is removed from an air line, preferably by a flow of air, and the field of view of the imaging system is dried in this way. This step is required in particular if the endoscope is used in a dry operation region. In a fourth step, the rotation drum is rotated back to the observation position, preferably the observation position from the first step.

The invention further relates to a method for operating an above-described endoscope with rotation drum and a pivotable bearing fork, wherein, in a first step, the shaft tube, at least the distal end of the shaft tube, is guided into an operation region and, in a second step, the bearing fork is pivoted about a second rotation axis, at least until the rotation drum exposes the work channel along a longitudinal axis of the shaft tube. In a third step, an instrument is guided from the work channel, in particular into the medical operation region. Subsequently and in a preferred fourth step, the rotation drum is pivoted by means of the control line about a first rotation axis in order to track the instrument in the operation region with a field of view of the imaging system and to image said instrument for an operator. In a fifth step, the bearing fork can be pivoted about a second rotation axis in order to move or bend the instrument and to align the latter with a defined operation region.

Preferably, the second sub-assembly has, on an outer face of the shaft tube, a displaceable handle, which is displaceable parallel to the longitudinal axis of the shaft tube, and wherein the handle has a recess for receiving a driver pin on the control line of the first sub-assembly.

The invention also relates to a method for using an already described hybrid endoscope, wherein at least the second sub-assembly, after production, is packed in a sterile state for sterile storage and is marked as a disposable part. The two sub-assemblies are then mounted in a sterile state directly before use, in particular for a medical operation, and are disassembled again thereafter. After disassembly, the first sub-assembly is treated in an autoclave and/or with a chemical liquid to permit renewed sterile use. The second sub-assembly is used once, disposed of and/or provided with a marking which indicates the previous use and preferably prevents an operator from joining it again to a first sub-assembly. For example, during the disassembly, it would be conceivable for the second sub-assembly to be damaged at a predetermined breaking point, preferably within the interface, in such a way that renewed assembly is excluded.

### BRIEF DESCRIPTION OF THE DRAWINGS

Further advantages and details of the invention will become clear from the following description of preferred embodiments of the invention and on the basis of purely schematic drawings, in which:

FIG. 1a shows a side view of a hybrid endoscope in an assembled state with a rotation drum, which is mounted in a shaft tube,

FIG. 1b shows a side view, as per FIG. 1a, of a first sub-assembly with a rotation drum, and an imaging system shown by dashed lines,

FIG. 1c shows a side view, as per FIG. 1a, of a second sub-assembly with the shaft tube,

FIG. 2a shows a side view of the hybrid endoscope in an assembled state, with a receiving device, the shaft tube and a rotatable joint,

FIG. 2b shows a side view, as per FIG. 2a, of the first sub-assembly,

FIG. 2c shows a side view, as per FIG. 2a, of the shaft tube with a rotatable joint.

The same elements, or elements with the same function, have been provided with the same reference signs in the figures.

### DETAILED DESCRIPTION

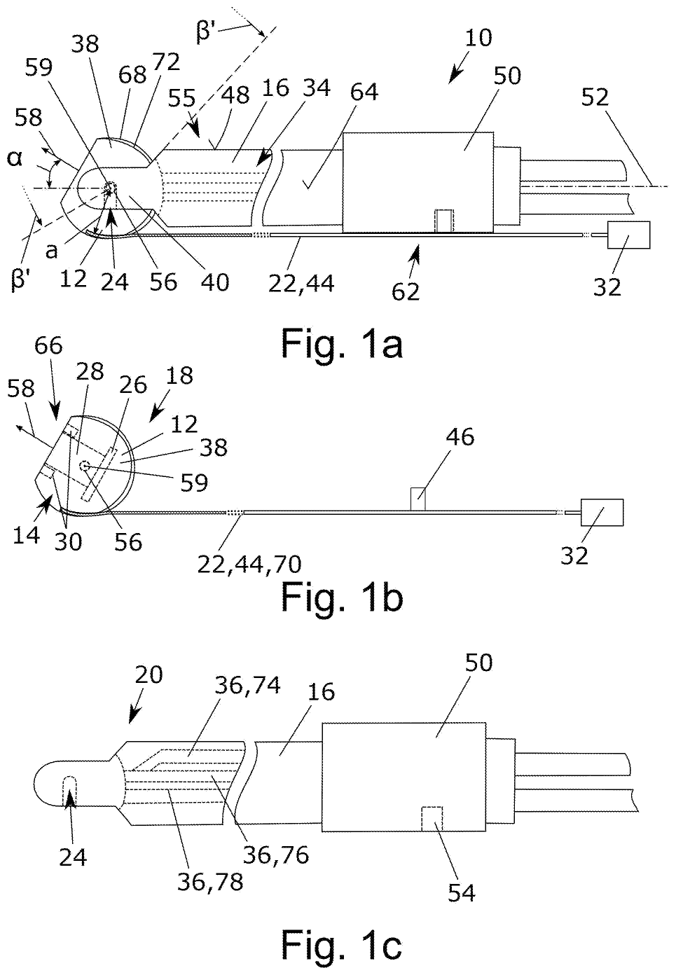

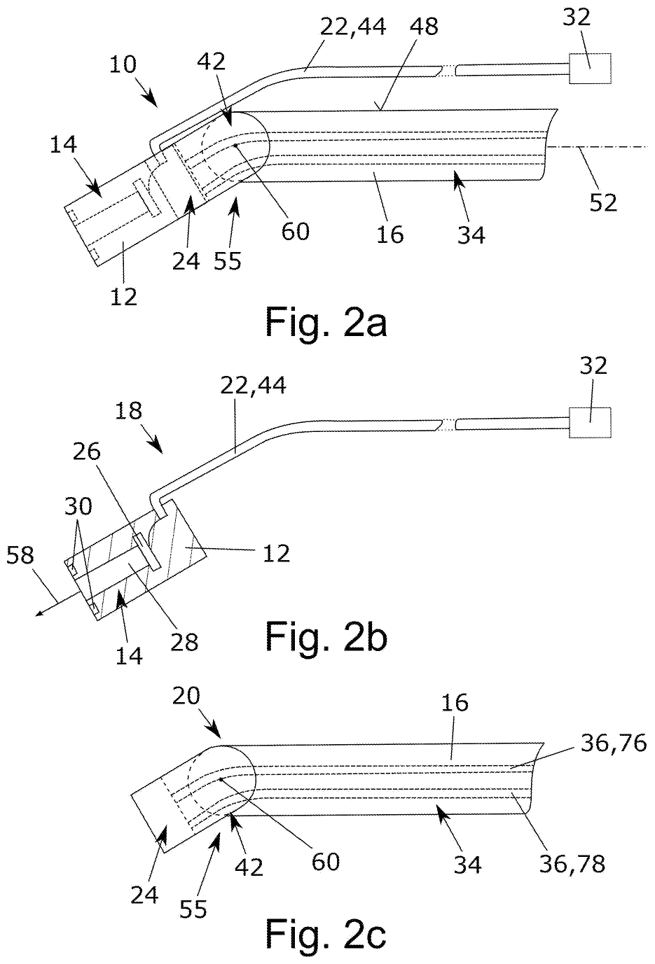

FIG. 1a and FIG. 2a each show a hybrid endoscope 10, in particular for a sterile medical application, wherein the endoscope 10 comprises a receiving device 12 for an imaging system 14 and a shaft tube 16. The hybrid endoscope 10 is shown in an assembled state in FIG. 1a and FIG. 2a, wherein the receiving device 12 is arranged at a distal end 55 of the shaft tube 16. The endoscope 10 is divided into two sub-assemblies 18, 20, each of which are shown individually in FIG. 1b and FIG. 1c and in FIG. 2b and FIG. 2c, respectively. FIG. 1b and FIG. 2b each show a first sub-assembly 18, which comprises at least the receiving device 12 and the imaging system 14, and also preferably a supply line 22 of the imaging system 14 as re-usable parts. FIG. 1c and FIG. 2c each show a second sub-assembly 20 which comprises at least the shaft tube 16 as a part for single use. As is shown in the mounted state in FIG. 1a and FIG. 2a, the two sub-assemblies 18, 20 can be brought into a releasable operative connection by means of an interface 24.

FIG. 1a and FIG. 2a show that the supply line 22 of the imaging system 14 preferably runs on an outer face 48 of the shaft tube 16. In this way, a work channel 34 in the interior of the shaft tube 16 is not restricted by this supply line 22. Furthermore, by virtue of the supply line 22 being routed along the outer face, the two sub-assemblies 18, 20 are particularly easy to assemble and disassemble, in particular being more easily releasable than a supply line 22 which is guided in a complex manner in the interior of the shaft tube 16.

Preferably, the parts of the first sub-assembly 18, in particular the receiving device 12, are hermetically sealed and thus autoclavable.

Particularly preferably, the receiving device 12 of the first sub-assembly 18 is produced from a metal, and the shaft tube of the second sub-assembly 20 is preferably produced from a plastic, and the supply line 22 is preferably connected to an autoclavable plug connector 32. This plug connector 32 is preferably connected to an attachment which is not in contact with the operation region and undergoes less complicated cleaning processes.

As is shown in FIG. 1b and FIG. 2b, the imaging system 14 can preferably comprise an electronic image recorder 26, an imaging optical unit 28 and an illumination device 30, in particular with LEDs. The imaging optical unit 28 of the imaging system 14 is oriented in a viewing direction 58, in order to detect in this viewing direction 58 an image on the image recorder 26.

Furthermore, in addition to or as an alternative to the imaging system 14, special sub-assembles such as laser projectors can be arranged in the receiving device 12.

Particularly preferably, the receiving device 12 of the first sub-assembly 18 is designed as a rotation drum 38, as per FIG. 1a and FIG. 1b, which at least receives the imaging system 14. As per FIG. 1a, the rotation drum 38 can be mounted in an interface 24 of a bearing fork 40 at the distal end 55 of the shaft tube 16 of the second sub-assembly 20, in particular mounted rotatably in this bearing fork 40, wherein the supply line 22 of the imaging system 14 serves as a control line 22, 44 of the rotation drum 38, and the rotation drum 38 preferably with bearing pins 56 can be pushed into and locked in the interface 22, preferably with form-fit engagement.

A first rotation axis 59 of the rotation drum 38 runs approximately transverse to a longitudinal axis 52 of the shaft tube 16, wherein the endoscope 10, as per FIG. 1a, has at least one, preferably one, control line 22, 44 on an underside 62 of the shaft tube 16. This at least one control line 22, 44 is routed on an outer face 48 of the shaft tube 16 and is fastened to the rotation drum 38 at a lever distance a from the first rotation axis 58. The rotation drum 38 is rotatable by a linear movement of the at least one control line 22, 44 parallel to the longitudinal axis 52 of the shaft tube 16.

The rotation drum 38 preferably has a spherical or cylindrical shape, with the rotation drum 38 preferably being flattened in a direction perpendicular to a viewing direction 58 of the imaging system 14 in order to receive the imaging optical unit 28 and the illumination device 30, preferably in the form of two LEDs, in a flattened region 66, as per FIG. 1b.

The at least one control line 22, 44 is preferably windable on at least one winding curve 68 along the outer circumference of the rotation drum 38. Particularly preferably, the at least one control line 22, 44 is windable in such a way that the rotation drum 38 is rotatable from a viewing direction 58 along the longitudinal axis 52 of the shaft tube 16 through at least an angle α of 0° to 180° and hence the viewing direction 58 is alignable with the interior of the shaft tube 16 and a work channel 34. The viewing angle β of the imaging system 14 is preferably 50° to 70°, particularly preferably 60°. As a result, a preferred observation region β′, in which the imaging system 14 can record the operation region by pivoting of the rotation drum 38 through an angle α, is between −35° and 215°, particularly preferably between −30° and 210°, wherein the angle of the observation region β′, just like the angle α, relates to a 0° direction along the longitudinal axis 52 of the shaft tube 16. For the purpose of pivoting the rotation drum 38, the at least one control line 22, 44 has an at least partially flexible configuration, in order to wind said control line on the at least one winding curve 68 of the rotation drum 38. In this case, the at least one winding curve 68 along the outer circumference can preferably have a circular or oval shape.

Particularly preferably, the at least one control line 22, 44 of the rotation drum 38 forms a supply line 22 for the imaging system 14, which is preferably designed as a flexible printed circuit board 70 illustrated in FIG. 1a or FIG. 1b, for supplying electronic circuits in the interior of the rotation drum 38. Particularly when using the flexible printed circuit board 70 as a control line 22, 44, the corresponding winding curve 68 is designed in such a way that the bending radius is greater than 0.5 mm. Alternatively, the imaging system 14 can also be electrically supplied by way of a cable.

The at least one control line 22, 44 is guided on the circumference of the rotation drum 38 in interlocking fashion, preferably in a depression 72, as per FIG. 1a and FIG. 1b, so that said control line is protected but at the same time the external diameter of the rotation drum 38 is not increased further, and so injury to tissue in an operation region can also be reduced.

In addition or alternatively, the second sub-assembly 20, as per FIG. 2a and FIG. 2c, has at the distal end 55 of the shaft tube 16 a rotatable joint 42 for fastening the receiving device 12. Preferably, the receiving device 12 shown in FIG. 2b can be fastened with supply line 22 to the rotatable joint 42, the supply line 22 serving as a control line 22, 44 of the rotatable joint 42.

This rotatable joint 42 can also be provided in addition to the embodiment in FIG. 1a, wherein the bearing fork 40 is configured as rotatable joint 42, and the rotation drum 38 is pivotable about a first rotation axis 59 and a second rotation axis 60 of the rotatable joint 42.

Preferably, the shaft tube 16 of the second sub-assembly 20 has at least one work channel 34. As per FIG. 1c or FIG. 2c, this work channel 34 can have at least one fluid line 36. Alternatively or in addition, an instrument (not shown) can be guided in the work channel 34.

An air line 36, 74 and/or preferably a liquid feed line 36, 76 and a liquid extraction line 36, 78 are preferably arranged in the work channel 34 of the shaft tube 16. The air line 36, 74 and the liquid lines 36, 76, 78 are shown in FIG. 1c and FIG. 2c. In an observation position, as per FIG. 1a and FIG. 2a, the air and/or liquid line 36 can be used for rear-side cooling of the imaging system 14.

Especially for cleaning the imaging system 22 of endoscopes 10 in a dry operation region, the rotation drum 38 is preferably rotatable to a cleaning position, wherein the rotation drum 38 with the viewing direction 58 is inclined in the direction of the work channel 34. In this cleaning position, contamination is preferably able to be rinsed from the flattened region 66 of the rotation drum 38, and hence a field of view of the imaging system 14 is cleanable, preferably by means of an air line 36, 74 in combination with the liquid feed line 36, 76, wherein the air line 36, 74 is preferably used to remove a rinsing liquid from the imaging system 14 and from the flattened region 66. Preferably, the rinsing liquid is removable using a liquid extraction line 36, 78, as a result of which an escape of rinsing liquid into a dry operation region can be minimized.

Preferably for endoscopes 10 with wet ambient conditions or in a wet operation region, the at least one fluid line 36 can be used to rinse an operation region in addition to the rear-side cooling and the cleaning of the rotation drum 38 or of the receiving device 12, in particular in order to rinse the operation region in the viewing direction 58 of the imaging system 14 in an observation position. For a medical application, a sterile fluid is preferably used to this end, for example processed air, oxygen or a physiological saline solution as liquid. However, an aqueous rinsing fluid is preferably used. The imaging system 22 can be cleaned simultaneously with the rinsing of the operation region, without having to pivot the rotation drum 38 to a cleaning position.

In a preferred embodiment as per FIG. 1a and FIG. 1c, the shaft tube 16 of the second sub-assembly 20 has, on an outer face 48, a displaceable handle 50. The handle 50 is displaceable parallel to the longitudinal axis 52 of the shaft tube 16 and preferably has a recess 54 for receiving a driver pin 46 of a control line 22, 44, in particular of the supply line 22 of the imaging system 14, of the first sub-assembly 18. By displacement of the handle 50, the rotation drum 38 is pivotable as per FIG. 1a, or also the receiving device 12 is pivotable as per FIG. 2a.

The invention also relates to a method for operating the aforementioned hybrid endoscope, wherein the two sub-assemblies 18, 20, as per FIG. 1a and FIG. 2a, are assembled in a sterile state directly before use, in particular for a medical operation, and then the first sub-assembly 18, as per FIG. 1b and FIG. 2b, is treated in an autoclave and/or with a chemical liquid to permit renewed sterile use, and the second sub-assembly 20, as per FIG. 1c and FIG. 2c, is used just once and therefore discarded.

The hybrid endoscope 10 described thus far can be modified in numerous ways without departing from the inventive concept. For example, it is conceivable to mark the two sub-assemblies 18, 20, in particular with a barcode or an electronic interface, such that, by means of an analog or electronic read-out, for example as soon as the imaging system 14 is electronically connected to a control unit, it is possible to ascertain whether one of the sub-assemblies 18, 20 is sterile or has already been used or cleaned, so as to prevent renewed use of an endoscope 10 that has already been used and is not sterile.

### LIST OF REFERENCE SIGNS

10 endoscope
12 receiving device
14 imaging system
16 shaft tube
18 first sub-assembly of the endoscope
20 second sub-assembly of the endoscope
22 supply line of the imaging system
24 interface
26 electronic image recorder
28 imaging optical unit
30 illumination device
32 autoclavable plug connector
34 work channel
36 fluid line
38 rotation drum
40 bearing fork
42 rotatable joint
44 control line
46 driver pin
48 outer face of the shaft tube
50 displaceable handle
52 longitudinal axis of the shaft tube
54 recess in the displaceable handle
55 distal end of the shaft tube
56 bearing pins of the rotation drum
58 viewing direction
59 first rotation axis
60 second rotation axis
62 underside of the shaft tube
66 flattened region of the rotation drum
68 winding curve
70 flexible printed circuit board
72 depression
74 air line
76 liquid feed line
78 liquid extraction line
a distance between the first rotation axis and the winding curve
α angle of the viewing direction of the imaging system
β′ observation region

1. A hybrid endoscope, for sterile medical applications, wherein the endoscope comprises a receiving device for an imaging system and a shaft tube, wherein the receiving device is arranged, in a mounted state, on a distal end of the shaft tube,
characterized in that the endoscope is divided into at least two sub-assemblies, wherein a first sub-assembly comprises at least the receiving device, the imaging system, and a second sub-assembly comprises at least the shaft tube as a part for single use, wherein the two sub-assemblies are able to be brought into releasable operative connection by means of an interface.

2. The hybrid endoscope as claimed in claim 1,
characterized in that the supply line of the imaging system runs on an outer face of the shaft tube.

3. The hybrid endoscope as claimed in claim 1,
characterized in that the parts of the first sub-assembly are hermetically sealed and therefore autoclavable.

4. The hybrid endoscope as claimed in, claim 1,
characterized in that the receiving device of the first sub-assembly is produced from a metal, and/or the shaft tube of the second sub-assembly is produced from a plastic, and/or the supply line is connected to an autoclavable plug connector.

5. The hybrid endoscope as claimed in claim 1,
characterized in that the imaging system contains an electronic image recorder and/or an imaging optical unit and/or an illumination device, in particular with LEDs, and/or special assemblies such as laser projectors.

6. The hybrid endoscope as claimed in claim 1,
characterized in that the receiving device of the first sub-assembly receives the imaging system in a rotation drum, and the second sub-assembly has an interface in a bearing fork in order to bear the rotation drum rotatably, wherein the supply line of the imaging system serves as at least a control line of the rotation drum.

7. The hybrid endoscope as claimed in claim 1,
characterized in that the second sub-assembly has, as interface, a rotatable joint with an adjustment mechanism for fastening the receiving device.

8. The hybrid endoscope as claimed in claim 1,
characterized in that the shaft tube of the second sub-assembly has at least one work channel for the guiding of at least one fluid line and/or of at least one instrument.

9. The hybrid endoscope as claimed in claim 6,
characterized in that the second sub-assembly has, on an outer face of the shaft tube, a displaceable handle, which is displaceable parallel to the longitudinal axis of the shaft tube, and wherein the handle has a recess for receiving a driver pin on the at least one control line of the first sub-assembly.

10. A method for using a hybrid endoscope, as claimed in claim 1, having the following steps:
production and subsequent sterile packaging of at least a second sub-assembly,
mounting of a first sub-assembly, on the second sub-assembly in a sterile state, in particular directly before use, in particular for a medical operation,
disassembly of the two sub-assemblies,
treatment of the first sub-assembly, for renewed sterile use, in an autoclave and/or with a chemical liquid,
disposal of the second sub-assembly and/or marking after use.
